(19) United States
(12) Patent Application Publication
(10) Pub. No.: US 2010/0076388 A1
(43) Pub. Date: Mar. 25, 2010

(54) Discharge device for pharmaceutical media

(76) Inventor: Miro Cater, Daytona Beach, FL (US)

Correspondence Address: FLYNN THIEL BOUTELL & TANIS, P.C., 2026 RAMBLING ROAD, KALAMAZOO, MI, 49008-1631, US

(21) Appl. No.: 12/284,243

(22) Filed: Sep. 19, 2008

(51) Int. Cl. A61F 9/00; A61B 5/11; A61B 3/10; A61B 3/14

(52) U.S. Cl. 604/298; 600/595; 351/221; 351/206

(57) ABSTRACT

A discharge device for pharmaceutical media has a reservoir for storing the medium, with a discharge opening for delivering the medium to the environment, the discharge opening in a use position being positioned in front of an eye of the user and a feed mechanism for feeding the medium from the reservoir to the discharge opening. A control system with a control unit and a sensor device is also provided. The control system is constructed for detecting an opening state of the eye of the user and/or a viewing direction of the eye of the user and for influencing the delivery of the medium as a function of the detected opening state and/or detected viewing direction.

### FIELD OF APPLICATION AND PRIOR ART

The invention relates to a discharge device for a pharmaceutical medium having a reservoir for storing the medium, with a discharge opening for delivering the medium to the environment and which in a use position is positioned in front of an eye of the user, and with a feed mechanism for feeding the medium from the reservoir to the discharge opening.

Such discharge devices are known from the prior art. They are used for introducing a medicament into an eye of the user. Medicaments which can be discharged using such discharge devices can e.g. be used for treating eye irritations.

As a function of the medicament which is to be discharged as the medium from the discharge device, to a varying extent importance is attached to a precise dosage of the medicament. In order to ensure a particularly precise dosage, the discharge process can take place in automated manner, e.g. using electrically operated pumping means.

Particularly in the case of such an automated discharge process it is, however, not desirable for the medium to strike the eyelid instead of the eye, e.g. when the user is blinking.

### Problem and Solution

The problem of the invention is to further develop such a discharge device with a view to an easy operation and/or a precise maintenance of the desired dosage.

According to the invention this is achieved in that the discharge device has a control system with a control unit and a sensor device, the control system being constructed for detecting an opening state of the eye of the user and/or a viewing direction of the eye of the user and to influence the delivery of the medium as a function of the detected opening state and/or detected viewing direction.

Thus, according to the invention, the state of the eye is detected and used for controlling the discharge process. This influencing can e.g. involve the discharge process being started or finished as a function of the detected state. Such influencing can also involve the brief interruption of a discharge process if, during said discharge process, the eye of the user is temporarily closed.

The control unit preferably has an electronic circuit constructed for evaluating the sensor device signals and as a function of the result of the evaluation influencing of the medium discharge takes place. To this end the control unit is preferably connected to one or more discharge device components influencing the discharge process.

With respect to the sensor device, preferably there are a source of electromagnetic radiation and a sensor, the electromagnetic radiation source being so oriented in a use position that with the eye open the radiation is at least partly reflected in the direction of the sensor device. The sensor of the sensor device is so adapted to the source that it is suitable for detecting the reflected electromagnetic radiation. The radiation delivered by the source is reflected by the cornea and/or corium of the eye and is detected by the sensor, which can be constructed as a simple photosensor. As the reflection characteristics of the closed and opened eye clearly differ from one another, on the basis of the intensity of the light detected by the sensor the control unit can draw conclusions regarding the opening state of the eye. It is also possible to detect whether there is a correct use position. Besides the opening state of the eye, using such a structure it is also possible to a limited extent to detect the viewing direction of the user, because the reflection characteristics of the cornea and corium of the eye also differ from one another.

To ensure a correct evaluation of the signals detected by the sensor, it is possible to construct the control system in such a way that in a calibration mode taking place prior to the actual discharge process and for a specific user to detect the reflected radiation in the opened and closed state of the eye in order to allow adaptation to said user, e.g. by fixing a threshold value adapted to said user.

It is considered particularly advantageous if the electromagnetic radiation source is positioned above the eye. Linked with the instruction to the user to look at the source during the discharge process, this ensures that the user opens particularly widely the eye in question, which is advantageous for a successful outcome to the discharge process.

The electromagnetic radiation source and corresponding sensor are preferably provided in a common structural unit, so that the relative position of the source and sensor are already fixed by said structural unit. Thus, during manufacture the corresponding module can be handled as a whole, so that problems with regard to a correct orientation of sensor and source do not arise.

With regards to the nature of the electromagnetic radiation delivered by the source, it is in particular possible to use visible light in the wavelength range between 380 and 780 aem. However, alternatively, the source can also be constructed for the delivery of electromagnetic radiation in the non-visible wavelength range, preferably in the infrared frequency range. The advantage of using non-visible radiation is that there is no need to fear user dazzling.

Alternatively to the particularly simple construction with an electromagnetic radiation source and a corresponding sensor, which can be constructed as a simple light sensor, e.g. as a photoresistor, photodiode, phototransistor or photoelectric cell, the sensor device can also have a camera oriented with the eye in the use position and whose pictorial information is evaluated by the control unit for determining the opening state and/or viewing direction.

The camera can have a very simple construction, because a high resolution is unnecessary for most applications. Evaluation preferably takes place by means of a pattern recognition performed in the control unit. The use of a camera allows a highly differentiated detection of the state of the eye, so that it is e.g. possible by modifying the viewing direction for the user to control the discharge device in planned manner, e.g. starting the discharge process by looking to the left and upwards and ending it by looking to the right and upwards. Obviously with this configuration a detection of the opening state is also reliably possible.

In a further development of the invention the discharge device has a status-LED, which is located in the visual range of the user in the operating position. As a result of said status-LED, which is preferably constructed for delivering light in different colours, it is possible to provide the user in a simple manner with use instructions. Thus, the status-LED can e.g. change its state if the discharge process is concluded, so that the user is made aware of this. The status-LED can also be used for calling on the user to briefly close his eye for relaxation and for the action of a partial charge of the medium.

A structural simplification can be achieved if the status-LED is constructed identically to the electromagnetic radiation source for detection by a corresponding sensor.

The discharge device control system is preferably constructed for starting a discharge process and/or briefly interrupting a discharge process as a function of the opening position and/or the viewing direction.

Thus, the control system can be designed for starting the discharge process as soon as the eye is opened following the positioning of the discharge device. A restarting of the discharge process following an interruption to allow the medium to act can be made dependent on whether the eye has been intermediately closed and reopened. It is considered particularly advantageous if the control system is constructed for briefly interrupting the discharge process as a function of the opening position. This prevents the medium striking the eyelid instead of the eye of the user during blinking.

For controlling the discharge process by the control system the latter is preferably constructed for influencing medium delivery by activating and/or deactivating the feed mechanism and/or by opening and/or closing a discharge valve associated with the discharge openings.

Activation or deactivation of the feed mechanism is particularly appropriate in the case of electrically operated feed mechanisms. Thus, e.g. following the discharge of a given medium quantity, the feed mechanism can initially be deactivated by switching off a pump motor, followed by a wait during which the user briefly closes his eye and then reopens it before the feed mechanism is reactivated. A design with a discharge valve transferable into an opened and/or a closed state by the control system is particularly suitable for control systems reactivating the user blinking. The discharge valve also allows a particularly rapid influencing, so that it is possible even after a few milliseconds or even shorter period of time to interrupt the medium flow. With a standard blinking duration of 50 to 500 milliseconds, the discharge of the medium on to the eyelid can be almost entirely prevented.

Influencing the delivery of the medium by opening/closing the discharge valve is also understood to cover influencing in such a way that a discharge valve opening and closing with a high frequency during the discharge process is kept in the closed state for the blinking period and then at the end of the blinking process returns to its high frequency opening and closing mode.

The discharge valve is preferably operated by means of an electromagnetically operating actuator or by means of a piezeoactuator, which ensures a particularly rapid discharge valve reaction.

In a further development of the invention the discharge device is constructed for medium delivery in the form of individual droplets. This design makes it possible to couple droplet delivery in planned manner with the prerequisite of the eye being open. The production of the individual droplets can in particular be ensured by said high frequency operating discharge valve, which e.g. opens and closes with a frequency of approximately 100 to 5,000 Hz. This repeated opening and closing process can be interrupted in the instant when a closed eye state is detected and persists until the eye is reopened. The dropwise medium delivery is also advantageous because as a result of the individual small droplets the eye does not close in reflex-like manner when the medium impacts.

### DETAILED DESCRIPTION OF AN EMBODIMENT

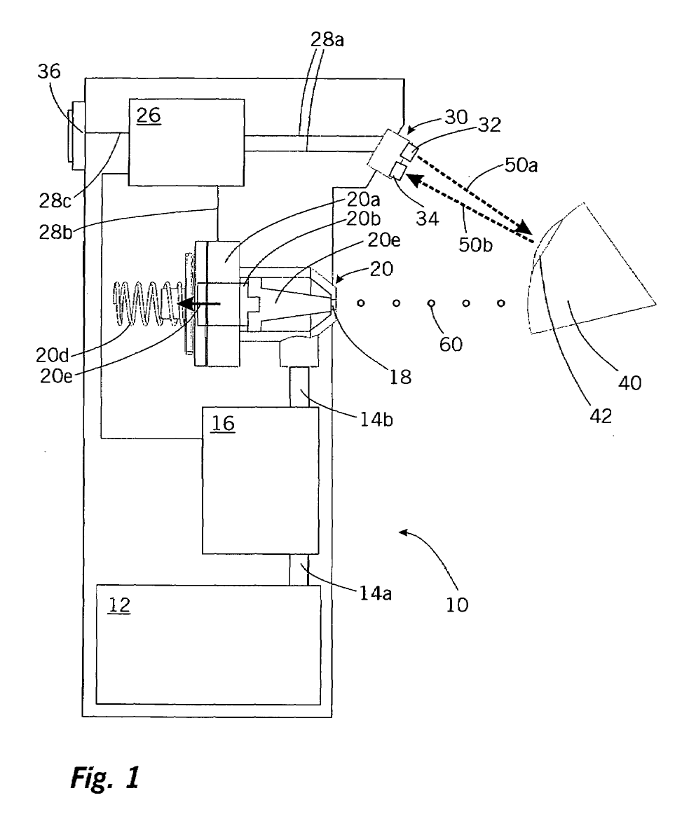

Further aspects and advantages of the invention can be gathered from the claims and the following description of a preferred embodiment of the invention illustrated in FIG. 1.

The discharge device 10 shown in FIG. 1 has a medium reservoir 12 for storing a liquid medicament. The medium can be fed through a channel 14a, 14b using a not shown pumping device 16 from medium reservoir 12 to a discharge opening 18. Between pumping device 16 and discharge opening 18 is formed a discharge valve 20, which can be controlled by means of a control coil 20a. Discharge valve 20 is constructed so that during an energizing of the control coil 20a a permanent magnetic core 20b is displaced in the direction of the arrow 20c counter to the spring force of a valve spring 20d, so that together with said core 20b a valve body 20e is displaced and consequently frees the discharge opening 18.

As further components discharge device 20 has a control unit 26 connected by lines 20a to a detector unit 30. Said detector unit 30 has a LED 32, which is constructed for delivering light or electromagnetic radiation in the non-visible range. Sensor unit 30 also has a photosensor 34. By means of control line 20a control unit 26 is able to control the light delivery by LED 32 and to detect the quantity of the light striking photosensor 34.

By means of a control line 28b control unit 26 is also connected to the coil 20a of discharge valve 20, so that the opening state of said discharge valve 20 can be controlled by means of control unit 26. By means of a third control line 28c control unit 26 is connected to an actuating key switch 36, which is provided on the outside of the discharge device.

The operation of the discharge device is described hereinafter.

Firstly the user moves the discharge device 10 up to his eye 40. As soon as discharge device 10 is in the correct use position, the user actuates key switch 36, so that control unit 26 receives the signal for starting the discharge process. Control unit 26 now controls the LED 32 so that the latter is illuminated green. In reaction thereto the user looks at said LED 32. This viewing direction leads to a characteristic reflection of the light 50a emanating from LED 32 onto the cornea 42 of the eye 40. The reflected light 50b strikes photosensor 34 and can therefore be detected by control unit 26. From said reflection control unit 26 concludes that the eye is opened and the viewing direction is correct. It therefore starts the discharge process in that it opens and closes in a rapid sequence discharge valve 20. This process, which can e.g. take place with approximately 500 Hz, leads to the medium being delivered in the form of individual droplets 60. Through a corresponding design of the feed mechanism 16 and discharge valve 20 it is possible to ensure that the droplets 60 always have roughly the same size, so that the discharge device can bring about a desired dosage by the delivery of a predetermined number of droplets.

If the user blinks during the discharge process and consequently his eye 40 closes, this is recorded by a change in the quantity of the reflected light 50b at sensor 34. As a reaction thereto, by a temporary closing of the discharge valve 20 control unit 26 interrupts the discharge process. Only when eye 40 has opened again and this is detected by the once again higher light incidence on sensor 34 is the discharge process continued until the desired medium quantity has been discharged.

The design described ensures that the medium completely or almost completely strikes the eye and is not delivered to the eyelid.

In the case of not shown designs of the discharge device in place of photosensor 34 use is made of a camera which is directed onto the eye of the user and detects its state. In this case the control unit 26 is constructed not only for evaluating an analog output signal of a photosensor, but also the detected camera pictorial data so that the opening state and/or viewing direction of the user is detected. As a result of such a design more complex control processes can be implemented where the viewing direction of the eye of the user can be used in planned manner for triggering discharge device functions.

The described discharge device can be equipped with a control unit 26, which interrupts the discharge process after a predetermined time or a predetermined number of droplets and requires the user through the status-LED, e.g. a blinking status-LED 32, to briefly close his eye so that the medicament can be received by it. In such a case the continuation of the discharge process is ordered by the control unit 26 as soon as the intermediate closure of the eye 40 and subsequent opening of the eye 40 has been detected.

The conclusion of the discharge process is revealed to the user by the LED 32, e.g. by the switching off of LED 32 or by a colour change of LED 32.

1. A discharge device for a pharmaceutical medium, said device comprising
a reservoir for storing the medium,
a discharge opening for delivering the medium to the environment, the discharge opening being positioned in front of an eye of a user in a use position
a feed mechanism for feeding the medium from the reservoir to the discharge opening, and
a control system comprising
a control unit and
a sensor device,
the control system
detecting an opening state of the eye of the user and/or a viewing direction of the eye of the user and
influencing the delivery of the medium as a function of the detected opening state and/or detected viewing direction.

2. The discharge device according to claim 1, additionally comprising
a source of electromagnetic radiation directed onto the eye in the use position and oriented in such a way that with the eye opened, the radiation is reflected in the direction of the sensor device and
a sensor associated with the sensor device for detecting the reflected electromagnetic radiation.

3. The discharge device according to claim 2, wherein the source delivers electromagnetic radiation in the non-visible wavelength range.

4. The discharge device according to claim 1, wherein the sensor device has a camera which is directed on to the eye and whose pictorial information is evaluated by the control unit for establishing the opening state and/or viewing direction.

5. The discharge device according to claim 1, additionally comprising a status-LED in the visual range of the user.

6. The discharge device according to claim 1, wherein the control system starts a discharge process and/or briefly interrupting a discharge process as a function of the opening position and/or viewing direction.

7. The discharge device according to claim 1, wherein the control system influences medium delivery by activating and/or deactivating the feed mechanism and/or opening and/or closing a discharge valve associated with the discharge opening.

8. The discharge device according to claim 7, wherein the discharge valve is operable by means of an electromagnetically operating actuator or by means of a piezoactuator.

9. The discharge device according to claim 1, wherein the discharge device delivers the medium in the form of individual droplets.

10. The discharge device according to claim 3, wherein the electromagnetic radiation is in the infrared frequency range.
